(19) United States
(12) Patent Application Publication
(10) Pub. No.: US 2004/0067495 A1
(43) Pub. Date: Apr. 8, 2004

(54) Polynucleic acid and polyamino acid binding substrate

(76) Inventor: Gustav Allen Ray, Pacifica, CA (US)

Correspondence Address: Gustav Allen Ray, 48 Desvio Court, Pasifica, CA, 94044, US

(21) Appl. No.: 10/266,161

(22) Filed: Oct. 8, 2002

(51) Int. Cl. C12Q 1/68

(52) U.S. Cl. 435/6

(57) ABSTRACT

A binding substrate comprising a water permeable layer which comprises at least one polymer and an effective amount of a polymeric mordant for binding DNA, RNA and proteins.

### CROSS REFERENCE TO RELATED APPLICATIONS

Not applicable

### STATEMENT REGARDING FEDERALLY SPONSORED RESEARCH OR DEVELOPMENT

Not applicable

### REFERENCE TO MICROFICHE APPENDIX

Not applicable

### BACKGOUND OF THE INVENTION

Binding substrates are an established means of irreversably or reversably binding inks, DNA, RNA and proteins.

In the case of inks, imaging devices such as ink jet printers and pen plotters are established methods for printing various information including photos and multicolor graphics. Presentation of such information has created a demand for ink-receptive binding substrates useful for commercial and non-commercial graphics. Imaging with the ink jet printer involves depositing ink on the surface of these binding substrates.

In the case of DNA, RNA and proteins well established capillary techniques such as Northerns and Sourtherns are used to transfer DNA, RNA, and proteins that have been electrophoretically separated by size and molecular weight in agarose and acrylamide gels to binding substrates.

In the case of DNA, RNA and protein the binding substrate commonly used is nitro-cellulose. One example of nitrocellulose is the ______ available from schliecher and schoeller. A significant drawback to using this substrate is its cost and brittle characteristics when dry. Further the nitro-cellulose substrate is highly flammable when exposed to sufficient heat. As a result, any heating of the substrate must be done in a vacuum to avoid oxidation of the substrate and possible ignition.

It is desirable that the surface of these binding substrates be dry and non-tacky to the touch, even after absorption of significant amounts of liquid soon after binding the inks, DNA, RNA and proteins. Transparent polymers containing polymeric mordants such as polyaminopropyl biguandine, polyhydroxmethyl biguandine and chlorhexidine that are capable of absorbing significant amounts of liquid while maintaining some degree of durability and transparency, are useful as a binding substrate.

Liquid-absorbent materials are well known. These materials comprise crosslinked polymeric compositions capable of forming continuous matrices for liquid absorbent semi-interpenetrating polymer networks. These networks are blends of polymers such as polyvinyl alcohol 325 available from Celanese Chemicals and polyvinyl pyrolidone PVP K90 available from ISP Corporation and hydroxymethyl metyacrylate (HEMA), wherein at least one of the polymeric components is crosslinked after blending to form a continuous network throughout the bulk of the material, and through which the uncrosslinked polymeric components are intertwined in such a way as to form a macroscopically homogenous composition. Such compositions are useful for forming durable, water permeable, binding substrates.

The present inventors have now discovered a cost effective polynucleic acid and polyamino acid receptive material, which when used as binding substrate yields improved shelf life after binding, themally stable substrates, and strong binding of DNA, RNA and proteins to the substrate. Even after exposed to elevated temperature, high humidity, changes in pH and running water.

Polymeric mordants are well known in the photographic sciences and normally comprise materials containing quaternary ammonium groups, or less frequently phosphonium groups.

U.S. Pat. No. 2,945,006 comprises mordants which are reaction products of aminoguanidine and carbonyl groups.

U.S. Pat. No. 4,695,531 discloses mordants in a light-sensitize silver halide element for radiographic use. A spectrally sensitized silver halide emulsion layer is coated on at least one side of a transparent case, and coated between the case and the silver halide emulsion layer is a hydrophilic colloid layer containing a water-soluble acid dye capable of being decolorized during the photographic process. This dye is associated with a basic polymeric mordant

### SUMMARY OF THE INVENTION

The invention provides an improved ink, DNA, RNA and protein receptive layer and receptive substrate which exhibits longer shelf life, better binding, better durability and significantly less costs.

The improved receptive substrates of the invention comprise a substrate bearing on at least one major surface thereof, a receptive layer comprising a receptive polymer and an effective amount of at least one polymeric mordant. Non limiting examples of effective polymeric mordants are polyaminopropyl biguandine, polyhydroxmethyl biguandine and chlorhexidine.

### BRIEF DESCRIPTION OF THE SEVERAL VIEWS OF THE DRAWING

Not applicable

### DETAILED DESCRIPTION OF THE INVENTION

Polymeric mordants useful in binding polynucleic and polyamino compounds onto receptive substrates of the present invention contain polyaminopropyl biguanidine, polyhydroxy methyl biguanidine and chlorhexidine.

The binding layer of the receptive substrate of the invention further comprises a polymeric material. Although at least one of the polymers present in the polymeric receptive material is preferably crosslinkable, the system need not be crosslinked to exhibit the improved longevity and durability. Further, such crosslinked systems have advantages for dry time, resistance to tearing and structural integrity.

Preferably the receptive layer comprises a polymeric blend containing at least one water-absorbing, hydrophilic, polymeric material and a polymeric mordant.

The water-absorbing hydrophilic polymeric material comprises homopolymers or copolymers of monomeric units selected from vinyl lactams, alkyl tertiary amino alkyl acrylates or methacrylates, alkyl quaternary amino alkyl acrylates or methacrytates, 2-vinylpyridine, hydroxy ethyl methylmethacrylates and 4-vinylpyridine. Polymerization of these monomers can be conducted by free-radical techniques with conditions such as time, temperature, proportions of monomeric units, and the like, adjusted to obtain the desired properties of the final polymer.

When desired, a polyethylene glycol can be added to the receptive layer for the purpose of making the receptive layer flexible. Lower molecular weight polyethylene glycols are more effective for flexibility while maintaining a low level of haze. Accordingly, it is preferred that the polyethylene glycol have a molecular weight of less than 4000 and most preferably of 600.

In a preferred embodiment, the receptive layer is a

(1) polyvinyl alcohol;

(2) PVP K90

(3) crosslinking agent

(4) polyaminopropyl biguandine

The amount of crosslinking agent such as aziridine to be used is preferably that amount that will react with 5 to 150 mole percent, preferably 25 to 90 percent, of the unreacted anhydride units of the polymer that forms the matrix. When the crosslinking agent is added in an amount capable of reacting with more than 100 mole percent of the unreacted maleic anhydride units, unreacted hydroxyalkyl moieties will remain as part of the crosslinked product.

While it is the primary function of the crosslinkable component to impart physical integrity and durability to the coating without adversely affecting the overall liquid absorbency of the coating, it is the primary function of the liquid-absorbent component to promote absorption of liquids. When aqueous liquids are to be absorbed the liquid-absorbent component must be capable of absorbing water, and preferably be water-soluble.

The ink-receptive layer can also include particulate material for the purpose of improving handling, binding and flexibility. Preferred particulate materials include polymeric beads, e.g., poly(methylmethacrylate), poly(stearyl methacrylate)hexanedioldiacrylate copolymers, poly(tetrafluoroethylene), polyethylene; starch and silica.

The binding layer can be applied to a porous or non-pourous backing by any conventional coating technique, e.g., deposition from a solution or dispersion of the resins in a solvent or aqueous medium, or blend thereof, by means of such processes as Meyer bar coating, knife coating, reverse roll coating, rotogravure coating, slot die and the like.

Drying of the ink-receptive layer can be effected by conventional drying techniques, e.g., by heating in a hot air oven at a temperature appropriate for the ink-receptive layer and the specific film backing chosen, infared, UV curing and the like.

To promote adhesion of the receptive layer to the backing for added durability and integrity of the receptive layer, it may be desirable to treat the surface of the backing with one or more primers, in single or multiple layers. Useful primers include those known to have a swelling effect on the backing polymer. Examples include halogenated phenols dissolved in organic solvents. Alternatively, the surface of the backing may be modified by treatment such as corona treatment or plasma treatment.

The primer layer, when used, should be relatively thin, preferably less than 2 micrometers, most preferably less than 1 micrometer, and may be coated by conventional coating methods.

Further, the binding layer can applied to a film backing that contains a release layer to facilitate removal of the polymeric receptive layer from the backing. Once the dried polymeric binding layer is removed from the backing it can be used directly to bind DNA, RNA and proteins.

Further the backing may be transparent, opaque, or translucent. The backing may be paper based for film based or any other material such as glass or metal. Preferably, the backing is a 100 micron pore sized paper. One specific example of a such a paper is ______ avaible from ______.

The following examples are for illustrative purposes, and do not limit the scope of the invention, which is that defined by the claims.

What is claimed is:

1. A poly nucleic acid binding substrate comprising a hydrophilic layer comprising a hydrophilic polymer and an effective amount of a polymeric mordant.

2. A receptive substrate according to claim 1 wherein said polymeric mordant comprises from about 1 part by weight to about 20 parts by weight of Polyaminopropyl biguandine.

4. A receptive substrate according to claim 1 wherein said polymeric mordant comprises from about 1 part by weight to about 20 parts by weight of Polyhexamethyl biguandine.

5. A receptive substrate according to claim 1 wherein said polymeric mordant comprises from about 1 part by weight to about 20 parts by weight of Chlorhexidine.

6. A polyamino acid binding substrate comprising a hydrophilic layer comprising a hydrophilic polymer and an effective amount of a polymeric mordant.

7. A method for binding polynucleic acids to a substrate with an effective abount of a polymeric mordant.

8. A method for binding polyamino acids to a substrate with an effective amount of polymeric mordant.
